(19) United States
(12) Patent Application Publication
(10) Pub. No.: US 2008/0233149 A1
(43) Pub. Date: Sep. 25, 2008

(54) Use of Erythropoietin for Enhancing Immune Responses and for Treatment of Lymphoproliferative Disorders

(75) Inventors: Moshe Mittelman, Petach-Tikva (IL); Drorit Neumann, Ramat Hasharon (IL)

Correspondence Address: BROWDY AND NEIMARK, P.L.L.C.;624 NINTH STREET, NW, SUITE 300, WASHINGTON, DC, 20001-5303, US

(73) Assignees: YEDA RESEARCH AND DEVELOPMENT CO. LTD., Rehovot (IL); MOR RESEARCH APPLICATIONS LTD., Petach-Tikva (IL)

(21) Appl. No.: 11/632,220

(22) PCT Filed: Jul. 12, 2005
(86) PCT No.: PCT/IL05/00740

§ 371 Date: Aug. 22, 2007

Related U.S. Application Data
(60) Provisional application No. 60/586,880, filed on Jul. 12, 2004.
(60) Provisional application No. 60/586,870, filed on Jul. 12, 2004.

(51) Int. Cl. A61K 38/22; A61K 39/145; A61K 39/165; A61K 39/12; A61K 39/29; A61P 35/00

(52) U.S. Cl. 424/209.1; 514/8; 424/234.1; 424/212.1; 424/219.1; 424/225.1

(57) ABSTRACT

The present invention relates to the use of erythropoietin (EPO) for enhancing immune responses and for the treatment of lymphoproliferative disorders, excluding multiple myeloma. Accordingly to the invention EPO may be administered with a viral or bacterial vaccine.

### FIELD OF THE INVENTION

The present invention relates to erythropoietin and the use thereof for inducing immunity or enhancing general immune response, and in the treatment of lymphoproliferative disorders, excluding multiple myeloma.

Abbreviations: ALL: acute lymphocytic leukemia; AML: acute myelogenous leukemia; BM: bone marrow; BRMs: biological response modifiers; CLL: chronic lymphocytic leukemia; CML: chronic myelogenous leukemia; DTP: diphtheria-tetanus-pertussis; EPO: erythropoietin; Hb: hemoglobin; MM: multiple myeloma; MMR: measles-mumps-rubella; MMR-Var: MMR-varicella; MOPC: mineral oil-induced plasmacytoma in BALB/c mice; PBL: peripheral blood leukocytes; PC: plasma cells; PLL: prolymphocytic leukemia; rHuEPO: recombinant human EPO.

### BACKGROUND OF THE INVENTION

EPO (erythropoietin) is a glycoprotein hormone produced chiefly by specialized cells in the kidneys in adult organisms. These cells are sensitive to the oxygen concentration in the blood, and increase the release of EPO when the oxygen concentration is low. Since oxygen is carried by red blood cells, too few red blood cells and, consequently, anemia, will result in erythropoietin release. EPO acts on stem cells in the bone marrow to stimulate erythropoiesis, thus increasing the production of red blood cells.

Deficient (or inefficient) EPO production relative to hemoglobin level is associated with certain forms of anemia such as anemia of end-stage renal disease and anemias of chronic disorders, chronic infections, autoimmune diseases, rheumatoid arthritis, AIDS, and malignancies. The measurement of EPO levels in human serum has clinical importance. Determination of the level of EPO in patient serum can be useful in distinguishing those anemias and polycythemias that are associated with decreased or increased EPO levels from those that are not. Demonstration of an inappropriately low level of serum EPO may suggest that an anemic patient may benefit from treatment with recombinant EPO.

EPO has been previously disclosed for use in the treatment of anemia, including anemia found in patients with kidney failure, anemia associated with cancer (EP 358463), anemia caused by malignant tumors (U.S. Pat. No. 4,745,099), and anemia caused by bone marrow dysfunction, or due to radiation exposure or administration of carcinostatic substance (JP 2632014).

Recombinant human EPO (rHuEPO) is widely used in the clinical practice for treatment of anemia in patients with end stage renal failure. Clinical trials have also been carried out achieving varying results in increasing hemoglobin (Hb) and ameliorating anemia associated with AIDS (Henry et al., 1992), chronic diseases (Schreiber et al., 1996), and various malignancies such as solid tumors, myelodysplastic syndromes and multiple myeloma (Ludwig et al., 1990, 1993a and 1993b; Spivak, 1994; Mittelman et al., 1992 and 1997; Cazzola et al., 1995).

In all the above-mentioned patent documents and medical literature EPO is indicated only for the treatment of anemia. Treatment of renal cell carcinoma with EPO has been described in two studies: Rubins (1995) described transient tumor regression in a single patient with renal cell cancer who had been treated with EPO for anemia, and Morere et al. (1997) reported a study of 20 patients with metastatic renal cell carcinoma who received rHuEPO: one patient achieved “complete anti-tumor response”, another patient demonstrated partial response and 2 minor responses were further observed. These isolated observations with renal cell cancer patients did not indicate nor suggest a broad use of EPO in other malignancies.

International PCT Application No. WO 99/52543 of the same applicants described the use of EPO for the treatment of cancer, excepting renal cancer. Although an extended list of types of cancer that could be treated with EPO, including chronic lymphocytic leukemia (CLL) and lymphomas, was provided in the description, in fact positive results of treatment with EPO were shown only in the animal model of multiple myeloma, and patents derived from said PCT application were granted by the European Patent Office (EP 1 067 955 B1) and in the U.S. application Ser. No. 09/647,761 (to be issued on Jun. 17, 2003, under U.S. Pat. No. 6,579,525) only for the treatment of multiple myeloma. Mittelman et al., 2001, describe that the use of rHuEpo induced tumor regression and antitumor immune responses in murine myeloma models.

### SUMMARY OF THE INVENTION

The present invention relates, in one aspect, to a pharmaceutical composition for enhancing immune responses comprising erythropoietin (EPO) and a pharmaceutically acceptable carrier.

In another aspect, the invention relates to the use of EPO for the preparation of a pharmaceutical composition for enhancing immune responses.

In a further aspect, the invention provides a method for enhancing immune responses, which comprises administering to an individual in need an effective amount of EPO.

In accordance with the present invention, EPO is used for enhancing immune responses towards antigens including those associated with infectious diseases caused by pathogenic organisms such as virus, bacteria, parasites or fungi. In this aspect, it may be administered with a viral or bacterial vaccine to boost its effect. The administration of the erythropoietin preparation may occur before, concomitantly or after the administration of the viral or bacterial vaccine.

In a further aspect, the invention provides a kit comprising one container comprising EPO and one container comprising a viral or bacterial vaccine such as BCG, cholera, diphtheria-tetanus- pertussis (DTP), hepatitis A, hepatitis B, influenza, lyme disease, measles, measles-mumps-rubella (MMR), MMR-varicella (MMR-Var), menningococcal vaccine, mumps, pneumococcal vaccine, poliovirus vaccine, rabies, rubella, tetanus-diphteria, typhoid, varicella, or yellow fever vaccine; and instructions for administration of EPO before, concomitantly or after the viral or bacterial vaccine.

The present invention relates, in one aspect, to a pharmaceutical composition comprising EPO and a pharmaceutically acceptable carrier, for the treatment of lymphoproliferative disorders, excluding multiple myeloma.

In another aspect, the invention relates to the use of EPO for the preparation of a pharmaceutical composition for the treatment of lymphoproliferative disorders, excluding multiple myeloma.

In a further aspect, the invention provides a method for the treatment of a patient with a lymphoproliferative disorder, which comprises administering to the patient an effective amount of EPO, wherein said patient is not one suffering from multiple myeloma.

Lymphoproliferative disorders is a group of malignant neoplasms of the lymphoreticular system characterized by the proliferation of lymphocytes and/or lymphoid tissues and/or lymph nodes and include lymphocytic leukemias, multiple myeloma and non-Hodgkin's lymphomas. The lymphoproliferative disorders that can be treated with EPO according to the invention include chronic lymphocytic leukemia (CLL), prolymphocytic leukemia (PLL), hairy cell leukemia, large granular lymphocyte leukemia, Waldenstrom's macroglubulinemia and non-Hodgkin's lymphomas.

Any form of biologically active erythropoietin may be used according to the invention. These forms of biologically active erythropoietin include, but are not limited to, recombinant erythropoietin and analogs as described in U.S. Pat. Nos. 5,441,868, 5,547,933, 5,618,698 and 5,621,080 as well as human erythropoietin analogs with increased glycosylation and/or changes in the amino acid sequence as those described in European Patent Publication No. EP 668351 and the hyperglycosylated analogs having 1-14 sialic acid groups and changes in the amino acid sequence described in PCT Publication No. WO 91/05867. Other forms of EPO that may be useful according to the invention are pegylated erythropoietin (peg-EPO) comjugates as described in U.S. Pat. No. 6,340,742 and U.S. Pat. No. 6,583,272, herein incorporated by reference in their entirety as if fully disclosed herein, and sustained-release EPOs (SR-EPO). In a most preferred embodiment, the erythropoietin is recombinant human erythropoietin.

In accordance with the present invention, EPO may be used alone for the treatment of the lymphoproliferative disorder or in conjunction with chemotherapeutic agents and/or other known anti-neoplastic therapies such as radiotherapy, monoclonal antibodies, e.g. herceptin, and immunotherapy as well as with biological response modifiers (BRMs), gene therapy and siRNA therapies.

### BRIEF DESCRIPTION OF THE DRAWINGS

FIG. 1 is a graph showing that EPO treatment prolongs survival of a subset of MOPC-315 bearing mice.

FIG. 2 is a graph showing statistical analysis of MOPC-315 tumor size of progressor mice in EPO- and albumin-injected groups, using the linear mixed effect model. This figure depicts the predicted rate of tumor growth, based on the data obtained in the experiment presented in FIG. 1. Statistical analysis demonstrates that the growth rate of the tumors in progressor mice injected with EPO is significantly (p<0.0001) slower than that of progressor mice injected with albumin.

FIGS. 3A-B show that EPO treatment results in elevation of endogenous (non pathological) λ light chain antibodies. FIG. 3A shows a Western blot analysis with anti-λ light chain of sera from MOPC-315 tumor injected mice. The solid and empty arrows point at endogenous and MOPC-315 derived λ light chains, respectively. The top two panels reflect EPO-treated mice, the lower two panels reflect albumin-treated mice. The first EPO injection is on day 9. FIG. 3B is a graph showing the quantification of endogenous λ light chain antibodies. Endogenous X light chain antibodies were quantified by densitometric analysis of Western blots (an example of which is shown in FIG. 2A) of sera from 9 EPO-treated progressors (black), 12 albumin-treated progressors (light grey), 2 albumin-treated regressors (dark grey) and 6 EPO-treated regressors (white). λ light chain levels 9 days after MOPC-315 injection are considered as 1 arbitrary unit, as a reference point, as this was the point of initiation of EPO or albumin injection. Standard errors of the mean are depicted.

FIG. 4 is a graph showing quantification of pathologic protein in MOPC-315 injected mice. Pathologic λ light chain antibodies were quantified by densitometric analysis of Western blots of sera from 9 EPO progressors (black), 12 albumin progressors (light grey), 2 albumin regressors (dark grey) and 6 EPO regressors (white). λ light chain levels 9 days after MOPC-315 injection are considered as 1 arbitrary unit, as this was the point of initiation of EPO or albumin injection. Standard errors of the mean are depicted.

FIG. 5 shows increased anti-MOPC 315 antibodies in EPO-treated mice. Cell lysates from MOPC-315 cells were resolved on SDS-PAGE and immunoblotted onto a nitrocellulose membrane filter which was subsequently probed with sera (diluted 1:100) obtained one month following MOPC-315 tumor cell injection from EPO (lane 2) and albumin (lane 4) treated mice (both mice were regressors with respect to tumor growth). Lanes 1 and 3 represent reactivity of sera from the respective mice before tumor cell injection. The higher reactivity of the serum in lane 2 compared to that of lane 4 can be noted.

FIG. 6 shows photographs of a leukemic BALB/c mouse bearing a BCL1 lymphatic tumor (right panel, lower part) and a healthy BALB/c mouse (right panel, upper part), and of their respective spleens (left panel). The sick mouse was obtained by injecting 10⁴ peripheral blood leukocytes (PBL) from a BCL1-bearing mouse displaying>80×10⁶cells/ml PBLs.

FIG. 7 is a graph showing the survival curve of BCL1-bearing mice, injected with EPO or albumin (control). Mice (15 in each group) were injected with 10⁴ PBLs from a BCL1-bearing mouse (PBL/BCL1) displaying>80×10⁶cells/ml PBLs. EPO injections were initiated 4 days following PBL/BCL1 injection.

FIGS. 8A-B: FIG. 8A is a graph showing the survival curve of BCL1-bearing mice, injected with EPO or albumin (control). Mice (14 in each group) were injected with 10⁴ BCL1 PBLs from a BCL1-bearing mouse displaying>80×10⁶cells/ml PBLs. EPO injections were initiated 19 days following BCL1 injection. FIG. 8B shows that EPO reduces tumor mass. PBL counts obtained 26 and 29 days following BCL1 injection (7 and 10 days following the initiation of EPO injections, respectively).

### DETAILED DESCRIPTION OF THE INVENTION

In the above-mentioned WO 99/52543, it has been disclosed that EPO was found to affect tumor regression in mice and to improve the biological and clinical course of multiple myeloma (MM) patients. The first observations were derived from MM patients being treated for anemia with EPO.

MM is characterized by a clonal proliferation of bone marrow (BM) transformed plasma cells (PC) secreting a paraprotein which can be detected in the serum and/or urine (Kyle, 1975; Durie & Salmon, 1975; Bergsagel, 1990). The mineral oil-induced plasmacytoma in BALB/c mice, designated MOPC-315 tumor, is a well-known murine model for the study of clinical and immunological aspects of human MM (Potter and Walters, 1973). Similarly to the human MM cells, the murine MOPC-315 tumor cells synthesize and secrete a monoclonal IgA (λ2) immunoglobulin, thereby providing a measurable tumor marker (serum myeloma component) during tumor progression. In WO 99/52543, the in vivo effect of rHuEPO treatment on the growth of MOPC-315 tumor cells was studied in BALB/c mice, and tumor regression was strikingly observed in 30-60% of mice challenged with MOPC-315 tumor cells and further treated for a short period with EPO, without tumor recurrence throughout a follow-up period of 3-7½ months. The event seems to be associated with the development of an effective antitumor immune response.

According to the present invention, the experiments of WO 99/52543 were repeated in another laboratory using BALB/c mice maintained under different conditions and, as described in Examples 1 and 2 hereinafter, tumor regression was again observed in 30-60% of mice challenged with MOPC-315 tumor cells and further treated for a short period with rHuEPO.

As used herein in the specification, “regressor mouse” or “regressor” refers to a mouse injected with tumor cells, followed by initial tumor growth and gross disappearance of the tumor cells after EPO treatment. “Progressor mouse” or “progressor” refers to a mouse injected with tumor cells, followed by continuous tumor growth irrespective of EPO treatment.

The results obtained according to the present invention with the progressor and regressor mice indicate that EPO may have a role in increasing protein production, particularly normal polyclonal immunoglobulin (Ig) production, and thus may have a beneficial effect in enhancing immune responses, for example towards antigens including those associated with infectious diseases caused by pathogenic organisms such as virus, bacteria, parasites or fungi. In this aspect, EPO may be useful to enhance the immune response when administered together with viral or bacterial vaccines such as BCG, cholera, diphtheria-tetanus-pertussis (DTP), hepatitis A, hepatitis B, influenza, lyme disease, measles, measles-mumps-rubella (MMR), MMR-varicella (MMR-Var), menningococcal vaccine, mumps, pneumococcal vaccine, poliovirus vaccine, rabies, rubella, tetanus-diphteria, typhoid, varicella, or yellow fever vaccine. EPO may be administered before, concomitantly or after administration of the vaccine.

According to the present invention, EPO, that has been used so far only for the treatment of anemia, including cancer-related anemia, and has been proposed for the treatment of multiple myeloma (MM) patients, was now found to reduce tumor mass and prolong survival of mice bearing BCL1-lymphoid malignancy.

BCL1 is a B-cell leukemia/lymphoma that developed spontaneously in a 2-year old female BALB/c mouse (Slavin and Strober, 1978). BCL1 is advantageous as an experimental model due to its analogies to human lymphocytic leukemias/lymphomas.

The leukemias and lymphomas are malignant tumors of the immune system. Leukemias are malignant neoplasms of blood-forming tissues. Leukemias tend to proliferate as single cells and are detected by increased cell numbers in the blood or lymph nodes. Leukemias can develop in lymphoid or myeloid lineage and were classified, historically, as acute lymphocytic leukemia (ALL), acute myelogenous leukemia (AML), chronic lymphocytic leukemia (CLL), and chronic myelogenous leukemia (CML). Lymphomas are a heterogeneous group of neoplasms arising in the reticuloendothelial and lymphatic systems. Lymphomas proliferate as solid tumors within a lymphoid tissue such as the bone marrow, lymph nodes, or thymus, and they include Hodgkin's disease and non-Hodgkin's lymphomas.

The results shown herein with the BCL1 mouse model indicate that EPO can be useful in the treatment of lymphoproliferative disorders including chronic lymphocytic leukemia (CLL), prolymphocytic leukemia (PLL), Waldenstrom's macroglubulinemia, hairy cell leukemia, lymphomas (non-Hodgkin's) and large granular lymphocyte leukemia.

EPO may be useful in the treatment of said lymphoproliferative disorders for inhibition of tumor growth, triggering of tumor regression, and/or inhibition of cancer cell metastasis.

According to the invention, EPO may be used alone for the treatment of the lymphoproliferative disorder or in conjunction with chemotherapeutic agents and/or radiotherapy and/or other known anti-neoplastic therapies such as monoclonal antibodies and immunotherapy as well as with biological response modifiers (BRMs), gene therapy and siRNA therapies.

As used herein in the specification and the claims, “erythropoietin” includes all types of erythropoietin, both natural and recombinant, as well as erythropoietin analogs showing erythropoietin activity, that are suitable for human administration such as the hyperglycosylated analogs and analogs having 1-14 sialic acid groups and changes in the amino acid sequence mentioned above. In one preferred embodiment, the erythropoietin is recombinant human erythropoietin (rHuEpo).

Any suitable route of administration of EPO such as intravenously (i.v.) or subcutaneously (s.c.), can be used according to the invention. The dose to be administered and the protocol of administration will be determined by the physician according to the severity of the disease, age and physical condition of the patient and other relevant parameters for each case. The present mice experiments suggest that the dose will be higher than the usual EPO dose for treatment of anemia.

The invention will now be illustrated by the following non-limiting Examples.

### EXAMPLES

### Materials and Methods

Materials

RHuEPO-α (Eprex) was obtained from Janssen-Cilag (Switzerland). RHuEPO-β (Recormon) was obtained from Roche. When rHuEPO containing albumin as carrier was employed, the control mice received human albumin (from Kamada Ltd., Beit Kama, Israel). When the rHuEPO preparation did not contain albumin, the control mice were injected with water.

The murine tumor plasmacytoma MOPC-315 was purchased from the American Type Cell Culture (ATCC), Rockville, Md., USA.

The BCL1 mouse B-cell leukemia/lymphoma cells were kindly provided by Prof. S. Slavin (Hadassah University Hospital, Jerusalem, Israel, and stored frozen until use. BCL1 cells can also be purchased from the American Type Cell Culture (ATCC), Rockville, Md., USA.

Animals

Female BALB/c mice, 6-8 weeks old, bred at the Tel-Aviv University animal facilities (Tel-Aviv, Israel), were used in all experiments.

Model of Myeloma

The mouse myeloma MOPC-315 was maintained in vivo by serial intramuscular (I.M) inoculation into syngeneic female BALB/c mice aged 8 weeks. BALB/c mice were injected subcutaneously (s.c.) with 10⁴ cells in the abdominal area. Local tumor growth (2-5 mm diameter) was observed 11-13 days after injection, gradually growing in size and causing death in 90-100% of mice 40-50 days after injection. Subcutaneous rHuEPO treatment was initiated when a tiny palpable tumor appeared at the site of injection. Each mouse was numbered and a follow-up of tumor size in individual mice was carried out every day during EPO administration (lasting usually 4 weeks). Tumor diameter (in mm) was measured using a vernier caliper.

Model of lymphoma

BALB/c mice were injected intraperitoneally (i.p.) with 10⁴ BCL1 cells. The appearance of the lymphoid neoplasm in mice was monitored by weekly peripheral blood leukocytes (PBL) counts. Clinical onset of the lymphoid malignancy is defined as PBL counts exceeding 20×10⁶ cells/ml.

The BCL1 cells were propagated and maintained in vivo by i.p. injection of 10⁴ PBLs (most of which are lymphocytes), obtained from the above BCL1-bearing BALB/c mice with advanced leukemia displaying more than 80×10⁶cells/ml PBLs, into syngeneic BALB/c mice. The appearance of lymphoid malignancy in the inoculated syngeneic mice was monitored by weekly PBL counts as above. PBL counts were determined by counting leukocytes in a blood sample drawn from the mice.

SDS Gel Electrophoresis (SDS-PAGE) and Western Blot Analysis

Western blot analysis was performed as previously described (Neumann et al., 1993). 2 μl of sera diluted 1:1 in 0.9% NaCl, were resolved on 10% SDS-PAGE. MOPC-315 immunoglobulin (20 μg) was loaded as a positive control. The gel was blotted onto nitrocellulose membrane filter, and probed with rabbit antibodies against mouse immunoglobulin λ light chain. Subsequently the blots were incubated with secondary antibody (donkey anti-rabbit IgG) coupled to horseradish peroxidase (HRP), and the bands were visualized using enhanced chemiluminescence (ECL) according to the manufacturer's instructions.

### EXAMPLES

### Example 1

### Treatment with EPO Prolongs the Survival of MOPC-315 Bearing Mice

The experimental system of the MOPC-315 tumor was set up as described in Materials and Methods above. Thirty mice (15 in each group) BALB/c were injected subcutaneously (s.c.) with 10⁴ MOPC-315 cells in the abdominal area. Local tumor growth (2-5 mm diameter) was observed 11-13 days after injection, gradually growing in size and causing death in 90-100% of mice 40-50 days after injection. Subcutaneous rHuEPO treatment was initiated when a tiny palpable tumor appeared at the site of injection. Each mouse was numbered and a follow-up of tumor size in individual mice was carried out every day during EPO administration.

Treatment with EPO was conducted as described in WO 99/52543, namely, 30 units of rHuEPO were injected s.c. in the back of MOPC-315-bearing mice, 9 days following tumor injection. As the EPO preparation contains high amounts of albumin (×1000 higher than the EPO), the control mice were injected with albumin alone using the same amount that was present in the EPO preparations.

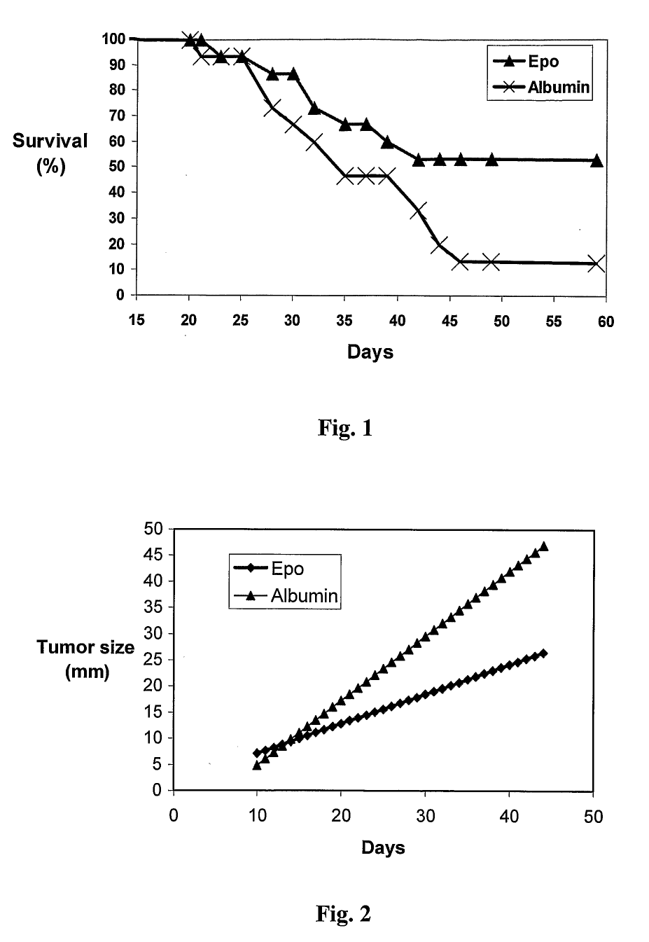

The results are summarized in FIG. 1. Survival of the MOPC-315 mice treated with EPO and control mice injected with albumin was followed for over a 60-day period. The results in FIG. 1 show that the EPO-mediated tumor regression was 30-60%. The median survival (MS) of the albumin-injected control group was 32 days. The MS of the EPO-treated mice cannot be calculated due to the survival of the subset group of regressor mice. These results confirm the results described in WO 99/52543 using experimental animals originating from a different animal facility and using a different control animal. The control mice of WO 99/52543 were injected water, while in the present experiments the control mice were injected with albumin. The survival curve of the progressor mice (a mouse injected with tumor cells, followed by continuous tumor growth) of the albumin-injected mice was similar to that of mice injected with diluent only, was similar (FIG. 1). Hence, the effect of rHuEPO on survival was observed on a subset of mice, the regressors.

### Example 2

### Treatment with EPO Slows the Rate of MOPC-315 Tumor Growth in Mice Compared to Tumor Growth in Albumin-injected Mice

While setting up the MOPC-315 system, we focused on the “progressor” mice with the following question in mind: Is the growth of the MOPC-315 tumor in the EPO-treated “progressor” mice similar to that of the control mice injected with albumin? Statistical analysis of the predicted rate of tumor growth, based on the data obtained in Example 1, shows that the tumor growth rate in EPO-treated progressor mice is significantly slower than that in albumin-injected progressor mice (FIG. 2). Thus, even in mice in which the tumor progresses, the progression in EPO-treated mice is slower, compared with the controls non-treated mice.

### Example 3

### EPO Treatment Results in Elevation of Endogenous (non-pathological) λ Light Chain

The mouse myeloma MOPC-315 cells synthesize and secrete immunoglobulin IgA with λ2 light chain and an α heavy chain. The molecular weight of this light chain is slightly lower than that of the endogenous λ light chain, thereby enabling to differentiate between the multiple myeloma derived λ light chain and the normal endogenous λ light chain by Western blot analysis. Sera from regressor or progressor mice in both EPO treated and control groups were collected at various time points. The serum born λ light chains (both endogenous and derived from the MOPC 315 cells) was resolved by SDS-polyacrylamide gel electrophoresis and detected by Western blot analysis with anti λ light chain antibodies.

40 mice were injected with 10⁴ MOPC-315 cells s.c. and after 9 days treated with either EPO or EPO diluting solution only. Sera was collected at various time points and analyzed by Western blots for λ immunoglobulin light chains.

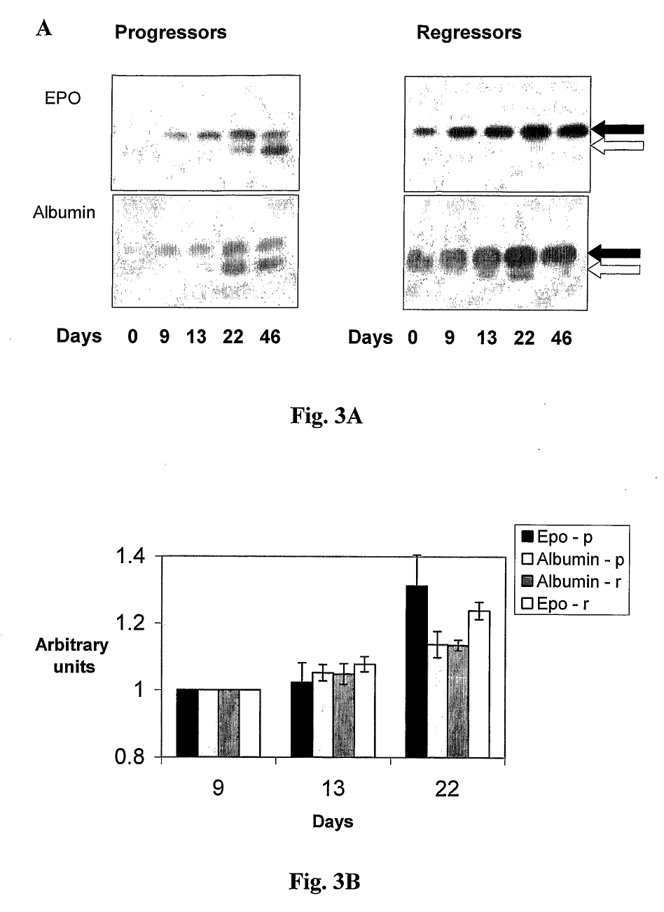

We monitored by Western blot analysis the levels of endogenous (non-pathological or normal polyclonal) λ immunoglobulin light chains, as well as the levels of the pathological λ light chain, which is derived from the MOPC-315 plasmacytoma cells. The four panels of FIG. 3A summarizes the results obtained with representative sera from MOPC-315 tumor injected mice. The solid and empty arrows point at endogenous and MOPC-315 derived λ light chains, respectively. The top two panels reflect EPO-treated mice, the lower two panels reflect albumin-treated mice. The first EPO injection was on day 9. The results depicted in FIG. 3A demonstrate that: (i) in the initial stages of the disease, in mice that eventually turned out to be progressors (left panels) or regressors (right panels), the levels of endogenous immunoglobulins are elevated, possibly due to an immune reaction against the myeloma cells (compare the upper bands, representing the endogenous λ light chain levels on days 0 and 9 following injection of MOPC-315 cells) (ii) In contrast to the regressors, the progressors show at later time points, at progressive states of the disease (by 46 days), decrease in the levels of endogenous immunoglobulin light chains decrease with respect to the pathological protein, reminiscent of the case in patients suffering from multiple myeloma.

FIG. 3B is a graph showing the quantification of endogenous λ light chain antibodies by densitometric analysis (using the TINA software program) of the Western blots (a representative Western blot is shown in FIG. 3A) of sera from 9 EPO-treated progressors (black), 12 albumin-treated progressors (light gray), 2 albumin-treated regressors (dark gray) and 6 EPO-treated regressors (white). The levels of λ light chain, 9 days after MOPC-315 injection are considered as 1 arbitrary unit, as this was the point of initiation of EPO or albumin injection. As shown in FIG. 3B, by day 22, EPO treatment resulted in a significant elevation of endogenous λ light chain in both progressor and regressor mice.

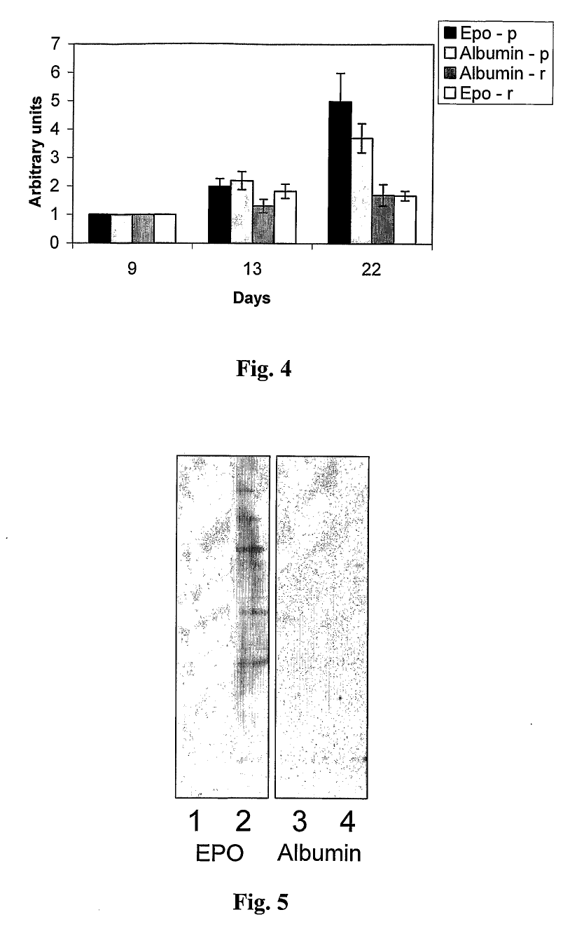

FIG. 4 is a graph showing the quantification of the pathologic protein in MOPC-315 injected mice. Pathologic λ light chain antibodies were quantified by densitometric analysis of Western blots of sera from 9 EPO-treated progressors (black), 12 albumin-treated progressors (light gray), 2 albumin-treated regressors (dark gray) and 6 EPO-treated regressors (white). The levels of λ light chain, 9 days after MOPC-315 injection are considered as 1 arbitrary unit, as this was the point of initiation of EPO or albumin injection. The levels of the pathological immunoglobulin correlated with the size of the tumor, and were reduced to minimum in the EPO- or albumin-treated mice in which the disease had undergone regression.

These results obtained, showing that EPO increases the levels of endogenous λ light chain, indicate that EPO may have a beneficial effect on enhancing immune responses towards other antigens in addition to MOPC-315 including common viral antigens used in vaccines, e.g. influenza, hepatitis, bacterial antigens and antigens against other infectious diseases used in vaccination.

### Example 4

### EPO Increases Anti-MOPC-315 Antibody Production in MOPC-315 Injected Mice

The preceding results show that EPO enhances the levels of the endogenous λ light chain, in the serum of EPO treated mice. The following analysis was carried out in order to explore whether EPO treatment also augments the levels of specific antibody production in MOPC-315 injected mice.

Lysates from MOPC-315 cells were resolved on SDS-PAGE and subjected to Western blot analysis detected with antisera obtained from an EPO- or an albumin-treated regressor mice. The results are summarized in FIG. 5. The results show that sera from both mice collected before tumor injection (lane 1 and 3) did not react with the MOPC-315 cell lysates in the blot. On the other hand, serum from the EPO-treated mouse (lane 2) displayed a higher reactivity with the MOPC-315 cell lysate compared to the serum from its albumin-treated counterpart (lane 4). Therefore, the results obtained indicate that the EPO treatment also augments the levels of specific antibody or enhance immunogenecity against an injected antigen such as MOPC-315 cells.

### Example 5

### Establishment of the BCL1 Lymphoma/Leukemia Mouse Model

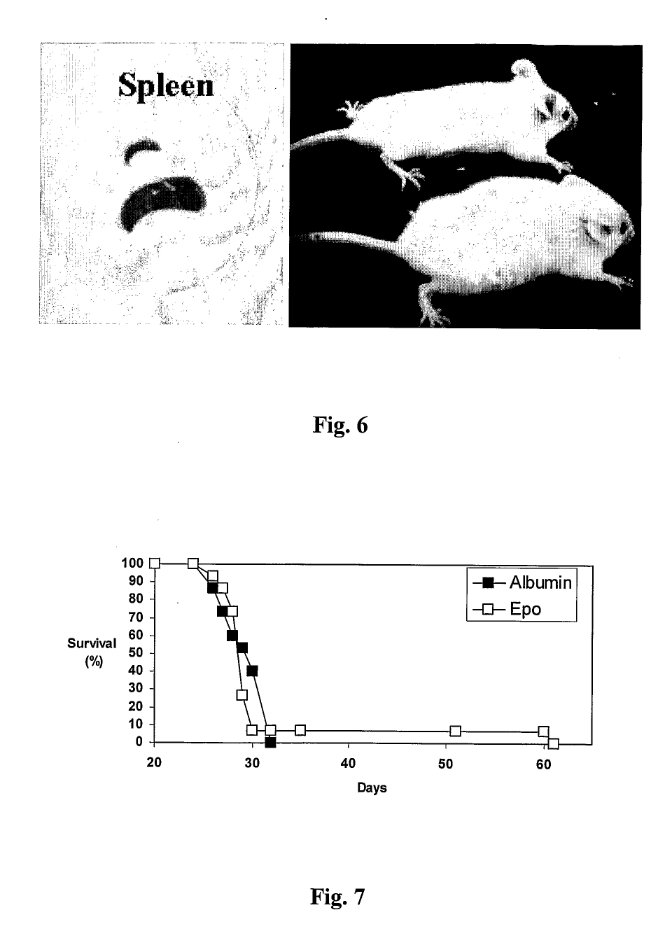

BALB/c mice were injected intra peritoneally (i.p.) with 10⁴ PBL cells from BCL1 tumor-bearing mice as described in Materials and Methods section. The course of the BCL1-lymphoma/leukemia disease was calibrated and it was found that the mice became sick during the fourth week following BCL1 injection. Mice were sacrificed and spleens were removed and examined at the terminal stage of the disease (21-28 days after injection). FIG. 6 , right panel, depicts photographs of a healthy (upper part) and a sick mouse (lower part) and their respective spleens (left panel). The results in FIG. 6 show that the sick mouse has a significantly larger spleen compared with the healthy mouse. The spleen weight and PBL counts of these two mice are shown in Table 1.

TABLE 1
Spleen weight and PBL count of
a BCL1 sick and a healthy mouse

| | Sick | Healthy |
|---|---|---|
| Spleen weight (g) | 1.83 | 0.16 |
| Leukocytes count (10⁶/ml) | 160 | 4 |

### Example 2

### EPO Reduces Tumor Mass and Prolongs Survival in the BCL1 Lymphoma/Leukemia Mouse Model

To assess the effect of EPO on BCL 1-bearing mice, two different regimens were tested (experiments 1 and 2 below).

Experiment 1: BALB/c mice (15 in each group) were injected i.p. with 10⁴ PBL cells from BCL1 tumor-bearing mice (as described in Materials and Methods) and the treatment with EPO started 4 days following the cell injection.

Experiment 2: BALB/c mice (14 in each group) were injected i.p. with 10⁴ PBL cells from BCL1 tumor-bearing mice (as described in Materials and Methods) and the treatment with EPO started 19 days following the cell injection.

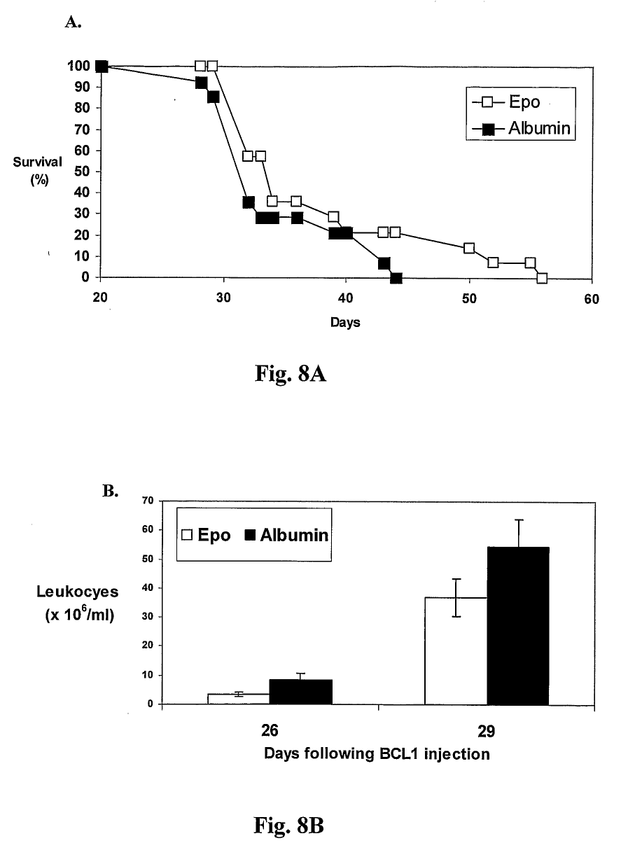

In both regimens, EPO was injected subcutaneously (30 units per mouse). In both cases, the injection regimens consisted of two courses of 5 daily EPO injections separated by a two days interval. This was followed by 3 weekly injections of EPO for two consecutive weeks. Since the EPO preparation contains high amounts of albumin (×1000 times higher than the EPO), the control mice in these experiments were injected with albumin alone (same amount as in the EPO preparation). The mice were followed for 2 months and the incidence of lymphoid malignancy and survival were recorded. The results of Experiments 1 and 2 are summarized in FIGS. 7 and 8, respectively. As shown in FIGS. 7 and 8 , EPO prolonged the survival of a small subset of mice up to 62 and 55 days, respectively, whereas all mice in the control groups died by 30 or 45 days (FIGS. 7 and 8A, respectively). FIG. 8B shows the PBL counts (reflecting tumor mass) obtained 26 and 29 days following BCL1/PBL injection (7 and 10 days following initiation of EPO treatment in experiment 2, respectively). The results summarized in FIGS. 8A and 8B show that in spite of the fact that the treatment with EPO is initiated later it appears to have therapeutic effect as manifested by the decrease in leukocyte counts (FIG. 8B) and increase in the cell survival (FIG. 8A) in comparison to control mice injected with albumin alone.

These results show that EPO reduces tumor load and prolongs survival in the BCL1 bearing mice.

### Example 6

### EPO Reduces Tumor Mass, as Indicated by Lower PBL Counts in BCL1-bearing Mice

In another experiment, BCL1-bearing mice were divided into three experimental groups. The control mice were injected with water, one group of mice were injected with EPO 15 days (dl5) following BCL1 injection, and another group of mice injected with EPO 19 days (d19) following BCL1 injection. The mice were bled 22 days following BCL1 injection (and after 7 days of EPO treatment in d15 and after 3 days of EPO treatment in d19) and their PBL counts were determined. As shown in Table 2, the EPO-treated mice had lower PBL counts in comparison to non-treated control mice. The results summarized in Table 2 also show that even thought the treatment with EPO is initiated later in the d19 group vis a vis the d15 group and in spite of its shorter duration (3 days versus 7 days in d19 versus d15, respectively) EPO has equally therapeutic effect as manifested by the decrease in leukocyte counts as compared to control mice injected with water alone.

TABLE 2
Effect of EPO on PBL counts (×10⁶/ml) in BCL1-bearing mice
Treatment

| EPO d19 | EPO d15 | Water |
|---|---|---|
| 57.6 | 10.4 | 44.6 |
| 6.8 | 22.4 | 13.2 |
| 9.8 | 12.8 | 35.2 |
| 7 | 16 | 38.4 |
| 6.6 | 5.2 | 28.8 |
| 5.6 | 5 | 48 |
| 9.3 | 8 | 7.8 |
| mean ± SEM | mean ± SEM | mean ± SEM |
| 14.7 ± 6.2 | 11.4 ± 2.1 | 30.9 ± 5.0 |

### REFERENCES

Abels R. Erythropoietin for anemia in cancer patients. Eur J Cancer 1993;29A(suppl 2):S2-8.

Cazzola M, Messinger D, Battistel V, Bron D, Cimino R, Enller-Ziegler L, Essers U, Greil R, Grossi A, Jager G, LeMevel A, Najman A, Silingardi V, Spriano M, van Hoof A, Ehmer B. “Recombinant human erythropoietin in the anemia associated with multiple myeloma or non-Hodgkin's lymphoma: dose finding and identification of predictors of response.” Blood. 1995 Dec 15;86(12):4446-53.

Henry D H, Beall G N, Benson C A, Carey J, Cone L A, Eron L J, Fiala M, Fischl M A, Gabin S J, Gottlieb M S, et al. Recombinant human erythropoietin in the treatment of anemia associated with human immunodeficiency virus (HIV) infection and zidovudine therapy. Overview of four clinical trials. Ann Intern Med. 1992 Nov 1;117(9):739-48.

Ludwig H, Fritz E, Kotzmann H, Hocker P, Gisslinger H, Bamas U. Erythropoietin treatment of anemia associated with multiple myeloma. N Engl J Med 1990;322:1693-9.

Ludwig H, Fritz E, Leitgeb C, et al. Erythropoietin treatment for chronic anemia of selected hematological malignancies and solid tumors. Ann Oncol 1993 Feb. 4:161-7. (2).

Ludwig et al. Europ J Cancer 29A (Suppl 2): s8-12, 1993 b.

Mittelman M, Floru S, Djaldetti M. Subcutaneous erythropoietin for treatment of refractory anemia in hematologic disorders. Blood. 1992 Aug 1;80(3):841-3.

Mittelman M. Recombinant erythropoietin in myelodysplastic syndromes: whom to treat and how? More questions than answers. Acta Haematol. 1993;90(2):53-7.

Mittelman M, Zeidman A, Fradin Z, Magazanik A, Lewinski UH, Cohen A. Recombinant human erythropoietin in the treatment of multiple myeloma-associated anemia. Acta Haematol. 1997;98(4):204-10.

Mittelman M, Neumann D, Peled A, Kanter P, Haran-Ghera N. Erythropoietin induces tumor regression and antitumor immune responses in murine myeloma models. Proc Natl Acad Sci U S A. 2001 Apr 24;98(9):5181-6. Epub 2001 Apr 17.

Morere J F, Bouillet T, Piperno-Neumann S, Tourani J M, Brunet A, Hennebelle F, Bareau J L. [Treatment of advanced kidney cancer using recombinant erythropoietin] Prog Urol. 1997 Jun;7(3):399-402. French.

Neumann D, Wikstrom L, Watowich S S, Lodish H F. Intermediates in degradation of the erythropoietin receptor accumulate and are degraded in lysosomes. J Biol Chem. 1993 Jun 25;268(18):13639-49.

Rubins J. Metastatic renal cell carcinoma: response to treatment with human recombinant erythropoietin. Ann Intern Med. 1995 May 1; 122(9):676-7.

Slavin S and Strober S. (1978), “Spontaneous murine B-cell leukaemia” Nature 272: 624-6.

Spivak J L. Recombinant human erythropoietin and the anemia of cancer. Blood. 1994 Aug 15;84(4):997-1004.

1-24. (canceled)

25. A method for enhancing immune responses which comprises administering to an individual in need an effective amount of erythropoietin.

26. The method according to claim 25 for enhancing immune responses towards antigens associated with infectious diseases caused by pathogenic organisms.

27. The method according to claim 26, wherein said pathogenic organisms are virus, bacteria, parasites or fungi.

28. The method according to claim 27, wherein the erythropoietin is administered together with a viral or bacterial vaccine such as BCG, cholera, diphtheria-tetanus-pertussis (DTP), hepatitis A, hepatitis B, influenza, lyme disease, measles, measles-mumps-rubella (MMR), MMR-varicella (MMR-Var), menningococcal vaccine, mumps, pneumococcal vaccine, poliovirus vaccine, rabies, rubella, tetanus-diphteria, typhoid, varicella, or yellow fever vaccine.

29. The method according to claim 28, wherein the erythropoietin is administered before said viral or bacterial vaccine.

30. The method according to claim 28, wherein the erythropoietin is administered concomitantly with said viral or bacterial vaccine.

31. The method according to claim 28, wherein the erythropoietin is administered after said viral or bacterial vaccine.

32. The method according to claim 25, wherein the erythropoietin is recombinant human erythropoietin.

33. The method according to claim 25, wherein the erythropoietin is hyperglycosylated and/or has changes in the amino acid sequence.

34. A method for treatment of a patient suffering from a lymphoproliferative disorder which comprises administering to said patient an effective amount of erythropoietin, wherein said patient is not one suffering from multiple myeloma.

35. The method according to claim 34, wherein said lymphoproliferative disorder is chronic lymphocytic leukemia (CLL), prolymphocytic leukemia (PLL), hairy cell leukemia, large granular lymphocyte leukemia, Waldenstrom's macroglubulinemia or a non-Hodgkin's lymphoma.

36. The method according to claim 34, wherein the erythropoietin is recombinant human erythropoietin.

37. The method according to claim 34, wherein the erythropoietin is hyperglycosylated and/or has changes in the amino acid sequence.

38. A kit comprising one container comprising erythropoietin (EPO) and one container comprising a viral or bacterial vaccine such as BCG, cholera, diphtheria-tetanus-pertussis (DTP), hepatitis A, hepatitis B, influenza, lyme disease, measles, measles-mumps-rubella (MMR), MMR-varicella (MMR-Var), menningococcal vaccine, mumps, pneumococcal vaccine, poliovirus vaccine, rabies, rubella, tetanus-diphteria, typhoid, varicella, or yellow fever vaccine; and instructions for administration of EPO before, concomitantly or after the viral or bacterial vaccine.
